(19) United States
(12) Patent Application Publication
(10) Pub. No.: US 2005/0065825 A1
(43) Pub. Date: Mar. 24, 2005

(54) X-ray computed tomography apparatus and operating method employing a computer program with patient-specific parameters

(76) Inventors: Dieter Boing, Muhlhausen (DE); Lutz Gundel, Erlangen (DE); Renato Tenorio Leite, Warfield (GB)

Correspondence Address: SCHIFF HARDIN LLP;Patent Department, 6600 Sears Tower, 233 South Wacker Drive, Chicago, IL, 60606, US

(21) Appl. No.: 10/903,638

(22) Filed: Jul. 30, 2004

(30) Foreign Application Priority Data
Jul. 31, 2003 (DE) 103 35 11 9.1

(51) Int. Cl. G06F 17/60; G21K 1/12; H05G 1/60; A61B 6/00; G01N 23/00

(52) U.S. Cl. 705/3

(57) ABSTRACT

In an x-ray computed tomography apparatus, and an operating method therefor, a data set containing patient-specific values is provided, a computer program to operate the x-ray computed tomography apparatus is provided, wherein parameters are assigned predetermined value, and the patient-specific values are adopted by the computer program, associated with the parameters corresponding thereto, and the predetermined values are overwritten with the patient-specific values.

### BACKGROUND OF THE INVENTION

1. Field of the Invention

The present invention concerns a method to generate patient-specific values for parameters in an x-ray computed tomography apparatus as well as an x-ray computed tomography,

2. Description of the Prior Art

In x-ray computed tomography, a distinction is made among the measurement of data, its pre-processing into raw data, and the subsequent computer-aided processing of the raw data into one or more images. The processing of the raw data is designated as reconstruction.

Dependent on the diagnosis requirement, it can be necessary to implement a number of measurements in succession, and under some circumstances the administration of contrast agent is also necessary. The measurements, the control of the contrast agent administration and the pre-processing and reconstruction ensue with the assistance of a computer according to predetermined programs. So that an optimally precise measurement, control and evaluation by means of the programs is possible, patient-specific values must be entered as inputs by the user for certain parameters. Such parameters can be age, gender, size, weight or the diameter of the patient. Reference is additionally made to Kalender, Willi “Computertomographie”, Publics MCD Verlag, 2000, in particular pg. 135-146.

From German OS 199 19 423, an x-ray computed tomography apparatus is known in which, dependent on the diagnosis requirement of the user, predetermined values are proposed for the parameters. The user has the possibility to change the predetermined values dependent on the details of the patient. Theoretically, the possibility exists of a modification by the user of the predetermined values leading to a failure of the x-ray computed tomography apparatus or even to damage to the x-ray tube. In order to prevent such an error function, the input values are automatically checked. In the event that an impermissible value combination is determined, the implementation of a measurement is automatically prevented. The user is additionally required to assign different values to the parameters. Nonetheless, in such known x-ray computed tomography apparatuses an input of incorrect values can occur that does not cause an error function. In such case, an unnoticeable faulty measurement can occur.

A computer-aided method to produce an operating protocol is known from PCT Application WO 02/39899. All events, values for parameters and data relevant in the production of an image by means of an x-ray computed tomography apparatus are in part automatically protocolled. A possibly wrong input of values for parameters can be determined by a precise testing of such a protocol. This is time-consuming and requires trained personnel.

### SUMMARY OF THE INVENTION

An object of the present invention is to avoid the aforementioned disadvantages of known systems. In particular, it is an object to provide a method and an apparatus with which a wrong measurement or the erroneous processing of the data of an x-ray computed tomography apparatus can be safely and reliably prevented.

This object is achieved according to the invention, by a method to generate patient-specific values for parameters in an x-ray computed tomography apparatus, the steps of providing a data set containing patient-specific values, providing a computer program to operate an x-ray computed tomography apparatus, wherein parameters are assigned predetermined values, and adopting the patient-specific values in the computer program, and in the computer program associating patient-specific the values with the predetermined parameters corresponding thereto, and overwriting the predetermined values with the patient-specific values.

With the inventive method, it is possible to implement a computer program to automatically operate an x-ray computed tomography apparatus using exact patient-specific values, allowing more precise results to be achieved.

A data set containing patient-specific values (for example size, age, gender and the like) is presently created for each patient in most medical treatment procedures and is stored in a network data processing system for use in the inventive method, therefore, such a data set need not be generated separately. It is most often already available. Conventional x-ray computed tomograph systems likewise themselves include a computer program for operation. In such a computer program, according to the prior art the parameters are assigned predetermined values. Thus, for example, one of a number of weight classes is predetermined for the weight of the patient. Dependent on of the weight of the patient to be examined, the user selects the appropriate one of the weight classes. The same takes place in a similar manner for the body size of the patient, etc. With the inventive method, exact values are now associated with the respective parameters necessary for operation of the x-ray computer tomograph. The values are—insofar as they are available—extracted from the already existing data set and are associated with the parameters corresponding thereto. Predetermined values associated with those parameters are overwritten with the exact values. Since other parameters are set with predetermined values, it is ensured (for example in the case of error) that the computer program still will be operated with somewhat reasonable values for the parameters.

In an embodiment, the data set containing patient-specific values is a component of a comprehensive overall data set forming the patient record. The data set, for example, can be extracted from the overall data set by the computer program, according to a predetermined algorithm.

The adoption, association and overwriting automatically can ensue by the computer program after creating a file for a further data set to store measurement results. In practice, a user first creates a further patient-specific data file for storage of the measurement results, in particular the acquired x-ray images. The creation of the file for the further data set ensues, for example, by entering the name of the patient. As soon as the name of the patient is entered, the associated overall data set is sought, from which the data set containing the patient-specific values is extracted by means of the computer program. The values are associated with the corresponding parameters, and predetermined values that already exist for the parameters are overwritten with the patient-specific values,

In a further embodiment, a value for at least one of the parameters is measured at the patient using the x-ray computed tomography apparatus. For example, the weight of the patient can be measured by means of a scale mounted in the bed of the x-ray computed tomography apparatus, and the measured weight can be directly associated with the program parameter corresponding thereto, it is also possible to optically measure the size of the patient by means of an optical detection device (for example a video camera, optical sensors or the like) provided at the x-ray computed tomography apparatus. It is also possible to provide pressure sensors in the bed, and therewith determine the size and/or the weight of the patient.

The values of the parameters can be displayed after the overwriting and can be used for measurement only after approval by the user. This enables a plausibility monitoring by the user. In the event that a value appears obviously senseless, a correction can ensue.

The parameters can include at least one of the following parameters: age, weight, gender, size, and existence of a pregnancy.

The parameters also can be combined in groups in the form of organ protocols. Organ protocols serve for the adjustment of the x-ray computed tomography apparatus with regard to the examination of specific predetermined organs.

According to a further embodiment feature, it is also possible that at least one further parameter is calculated from at least one other parameter using a predetermined algorithm. The algorithms are advantageously chosen dependent on the selection of the respective organ protocol. Moreover, given a change of a parameter group or of an organ protocol, one or more algorithms associated with these parameter groups is automatically changed. In this case, the system is adaptive: it automatically adapts to modified requirements.

The above object also is achieved in accordance with the invention by an x-ray computed tomography apparatus having a source for a data set containing patient-specific values, a memory in which a computer program is stored for operation of the x-ray computed tomography apparatus, wherein predetermined values are assigned to parameters, and a computer for executing the computer program that adopts the patient-specific values associates the patient-specific values with the parameters corresponding thereto, and overwrites the predetermined values with the patient-specific values.

Such an x-ray computed tomography apparatus enables a simplified and improved measurement. The x-ray images acquired thereby are particularly exact.

In embodiments of the x-ray computed tomography apparatus, for features explained above for the method are employed.

The aforementioned computer program can itself perform one or more of the following:
adopt the patient-specific values, associate the patient-specific values with the parameters corresponding thereto, overwrite the predetermined values, automatically adopt, associate and overwrite given creation of a further data set, control measurement of at least one value for one of the parameters, adopt the measured values, for association with the respective parameters, and for overwriting a predetermined value, control display of the values of the parameters after the overwriting, and allow approval by a user of an overwritten value.

The implementation of the aforementioned functions as a computer program can be alone relatively simply and cost-effectively.

### DESCRIPTION OF THE DRAWINGS

FIG. 1 schematically illustrates the basic components of an x-ray computed tomography apparatus.

FIG. 2 shows the basic components of a computer program to operate the x-ray computed tomography apparatus according to FIG. 1.

FIG. 3 shows the basic steps of the inventive method.

### DESCRIPTION OF THE PREFERRED EMBODIMENTS

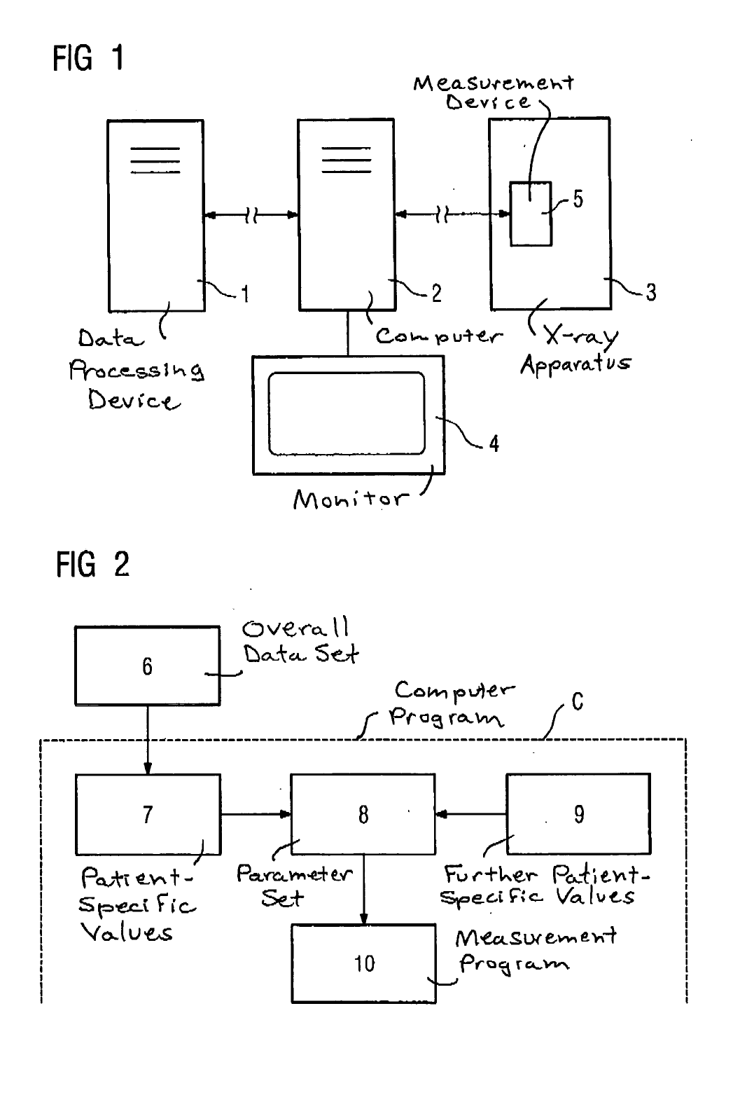

In FIG. 1, a data processing device 1, for example a central computer of a hospital, is connected with a computer 2 of an x-ray computed tomography apparatus. The x-ray computed tomography apparatus has an x-ray apparatus 3, and a monitor 4 connected to the computer 2. The x-ray apparatus 3 has a measurement device 5. The measurement device 5 can include sensors to detect patient-specific values for parameters, for example weight, size, topography and the like. The measurement device 5 is connected with the computer 2 for data transmission.

FIG. 2 shows the basic components of a computer program to operate the apparatus according to FIG. 1. A first data set 6 containing the patient-specific values is transferred from the data processing device to the computer program C via a suitable interface. By means of the computer program C, predetermined patient-specific values 7 are extracted from the first data set 6. The patient-specific values 7 are transferred to a predetermined parameter set 8. Each parameter is associated with the patient-specific value corresponding thereto. If necessary, the values previously associated with the parameters are overwritten. Further patient-specific values 9 that have been acquired by the measurement device 5 likewise can be transferred to the parameter set 8, and the corresponding values can be applied to the parameters corresponding thereto. Values previously assigned to the parameters can also thereby be overwritten. The parameter set 8 is finally transferred to a program 10 that controls the measurement of the x-ray computed tomography apparatus.

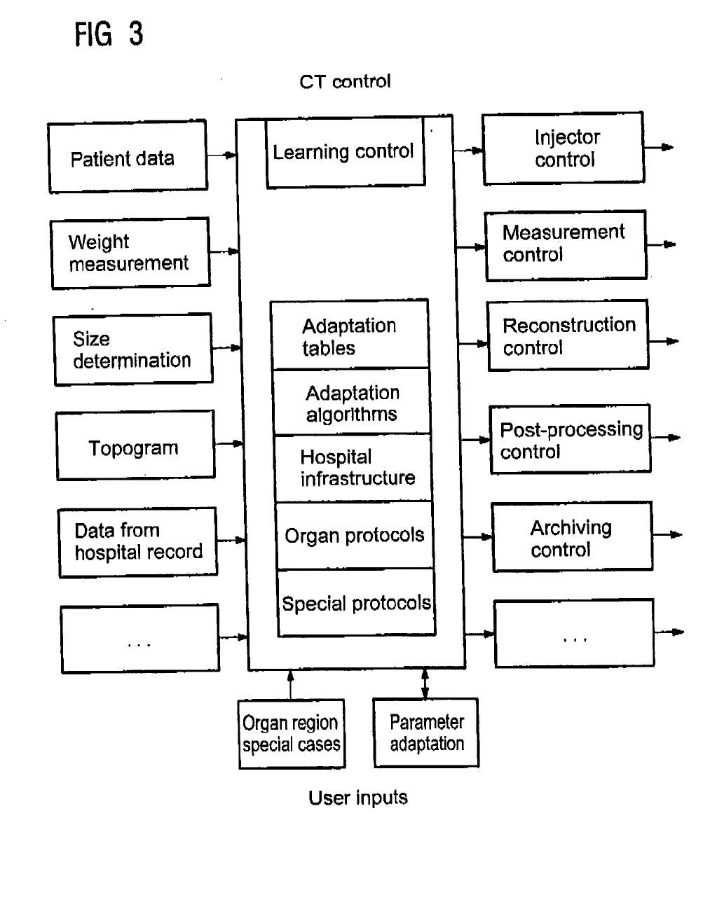

The significant steps and components of the inventive method are schematically shown in FIG. 3. External data sets and values that can be automatically adopted by the computer program (shown with its components in the center column) are shown in the left column. They are “patient data”, the value of a “weight measurement”, the value of a “size determination”, data about the “topogram” of the patient, and further “data from the hospital record”. The weight measurement, size determination and the determination of the topogram can ensue using suitable measurement devices such as the device 5 are provided at the x-ray computed tomography apparatus.

The transferred values are assigned to the patient-specific parameters by the computer program. Functions executed by the computer program, for example an “injection control”, a “measurement control”, a “reconstruction control”, a “post-processing control” and an “archiving control” then can be automatically implemented in adaptation to the patient-specific values for the parameters. Thus, for example, an amount of an administered contrast agent by means of the injection control can be precisely adapted to the patient. The further functions also can be exactly adjusted to the patient.

The adaptations ensue by means of further routines and data contained in the computer program, such as “adaptation tables”, “adaptation algorithms”, “organ protocols” and “special protocols”. The corresponding measurement protocols, such as organ protocols, special protocols, are adapted to the conditions specified by the patient-specific values for the parameters by means of the “adaptation algorithms”, using the “adaptation tables”.

Although modifications and changes may be suggested by those skilled in the art, it is the intention of the inventors to embody within the patent warranted hereon all changes and modifications as reasonably and properly come within the scope of their contribution to the art.

1. A method for generating patient-specific values for operating parameters for an x-ray computed tomography apparatus, comprising the steps of:
providing a date set comprising a pluralty of patient-specific values respectively for operating parameters for an x-ray computed tomography apparatus;
providing a computer program for operating said x-ray computed tomography apparatus in an examination of a patient to whom said patient-specific values apply, said computer program containing operating parameters with predetermined values assigned thereto;
opening a file for a further data set for storing measurement results of said examination; and
making said data set comprising said patient-specific values available to said computer program and in said computer program, after opening said file for said further data set, automatically adopting said patient-specific values and associating said patient-specific values with respective ones of said operating parameters, and overwriting said predetermined values for said respective ones of said operating parameters with said patient-specific values.

2. A method as claimed in claim 1 comprising providing a comprehensive overall data set forming an electronic patient record for said patient, and extracting said data set comprising said patient-specific values as a subset of said comprehensive overall data set.

3. A method as claimed in claim 1 comprising measuring a measured value for at least one of said patient-specific values using said x-ray computed tomography apparatus with the patient therein.

4. A method as claimed in claim 3 comprising, in said computer program, automatically adopting said measured value and associating said measured value with one of said operating parameters and overwriting said predetermined value for said one of said operating parameters with said measured value after measurement thereof using said x-ray computed tomography apparatus.

5. A method as claimed in claim 1 comprising, after overwriting said predetermined values with said patient-specific values, visually displaying the respective operating parameters and using the respective operating parameters for said examination only after an electronic input to said x-ray computed tomography apparatus indicating approval by a user.

6. A method as claimed in claim 1 wherein the step of providing said data set comprising patient-specific values comprises providing a data set comprising at least values respectively indicating age, weight, gender, size and existence of pregnancy of the patient.

7. A method as claimed in claim 1 comprising, in said computer program, combining said operating parameters in respective groups as organ protocols for examining respectively different organs.

8. A method as claimed in claim 1 comprising, in said computer program, calculating at least one further operating parameter from at least one of said operating parameters for which the respective predetermined values have been overwritten by said patient-specific values, using a predetermined algorithm.

9. A method as claimed in claim 8 comprising, in said computer program, combining said operating parameters in respective groups as organ protocols for examining respectively different organs, providing a plurality of algorithms respectively for said different organ protocols, and selecting one of said organ protocols for said examination and using the algorithm associated with the selected organ protocol as said predetermined algorithm.

10. A method as claimed in claim 9 comprising changing selection of said organ protocol from an initially selected organ protocol to a subsequently selected organ protocol, and in said computer program automatically changing said predetermined algorithm from the algorithm respectively associated with said initially selected organ protocol to the algorithm respectively associated with said subsequently selected organ protocol.

11. A medical examination apparatus for generating patient-specific values for operating parameters therefor, comprising:
an x-ray computed tomography apparatus;
a source for a data set comprising a plurality of patient-specific values respectively for operating parameters for said x-ray computed tomography apparatus; and
a control unit for executing a computer program for operating said x-ray computed tomography apparatus in an examination of a patient to whom said patient-specific values apply, said computer program containing operating parameters with predetermined values assigned thereto, said control unit when executing said computer program, opening a file for a further data set for storing measurement results of said examination, and said computer program having access to said data set comprising said patient-specific values said control unit, when executing said computer program after opening said file for said further data set, automatically adopting said patient-specific values and associating said patient-specific values with respective ones of said operating parameters, and overwriting said predetermined values for said respective ones of said operating parameters with said patient-specific values.

12. A medical examination apparatus as claimed in claim 11 comprising an electronic patient record for said patient containing a comprehensive overall data set accessible by said control unit, and said control unit, when executing said computer program, extracting said data set comprising said patient-specific values as a subset of said comprehensive overall data set.

13. A medical examination apparatus as claimed in claim 11 wherein said control unit operates said x-ray computed tomography apparatus with the patient therein for measuring a measured value for at least one of said patient-specific values.

14. A medical examination apparatus as claimed in claim 13 wherein said control unit, when executing said computer program, automatically adopts said measured value and associates said measured value with one of said operating parameters and overwrites said predetermined value for said one of said operating parameters with said measured value after measurement thereof using said x-ray computed tomography apparatus.

15. A medical examination apparatus as claimed in claim 11 comprising a display and an input unit connected to said control unit, and wherein said control unit, when executing said computer program and after overwriting said predetermined values with said patient-specific values, visually displays the respective operating parameters on said display and permits use of the respective operating parameters for said examination only after an electronic input via said input unit indicating approval by a user.

16. A medical examination apparatus as claimed in claim 11 wherein said source of said data set comprising patient specific values comprises a source of a data set comprising at least values respectively indicating age, weight, gender, size and existence of pregnancy of the patient.

17. A medical examination apparatus as claimed in claim 11 wherein said control unit, when executing said computer program, has access to combinations of said operating parameters in respective groups as organ protocols for examining respectively different organs.

18. A medical examination apparatus as claimed in claim 11 wherein said control unit, when executing said computer program, calculates at least one further operating parameter from at least one of said operating parameters for which the respective predetermined values have been overwritten by said patient-specific values, using a predetermined algorithm.

19. A medical examination apparatus as claimed in claim 18 wherein said control unit, when executing said computer program, has access to combinations of operating parameters in respective groups as organ protocols for examining respectively different organs, and access to a plurality of algorithms respectively for said different organ protocols, and wherein said control unit, when executing said computer program, selects one of said organ protocols for said examination and uses the algorithm associated with the selected organ protocol as said predetermined algorithm.

20. A medical examination apparatus as claimed in claim 19 comprising an input unit allowing changing selection of said organ protocol from an initially selected organ protocol to a subsequently selected organ protocol, and wherein said control unit, when executing said computer program, automatically changes said predetermined algorithm from the algorithm respectively associated with said initially selected organ protocol to the algorithm respectively associated with said subsequently selected organ protocol.
